(19) United States
(12) Patent Application Publication
(10) Pub. No.: US 2025/0164956 A1
(43) Pub. Date: May 22, 2025

(54) METHOD OF OPERATING A BIOPROCESS ARRANGEMENT TO PERFORM AT LEAST ONE REPETITION OF A BIOPROCESS

(71) Applicant: Sartorius Stedim Biotech GmbH, Goettingen (DE)

(72) Inventor: Jonathan Cutting, Heber City, UT (US)

(21) Appl. No.: 18/518,102

(22) Filed: Nov. 22, 2023

(51) Int. Cl. G05B 19/042

(52) U.S. Cl. CPC G05B 19/0428; G05B 2219/21039; G05B 2219/2243

(57) ABSTRACT

A method of operating a bioprocess arrangement to perform repetition of a bioprocess, wherein the bioprocess arrangement comprises a replaceable electronic component, wherein a process control system controls the bioprocess arrangement, using a digital model. The replaceable electronic components are removed and replaced by new electronic components, the process control system automatically detects the removal of the electronic components, automatically detects the presence of the new electronic components and retrieves digital representations of the new components, the process control system derives from the digital representations of the replaceable components and the new components a functional relationship, the process control system replaces the digital representations of the replaceable components with the digital representations of the new components based on the functional relationship thereby updating the digital model, and, that the process control system controls the bioprocess arrangement to perform the bioprocess based on the updated digital model.

### FIELD OF THE TECHNOLOGY

Various embodiments relate to a method of operating a bioprocess arrangement to perform at least one repetition of a bioprocess, to an electronic component and to a process control system.

### SUMMARY

The term “bioprocess” presently represents any kind of biotechnological process, in particular biopharmaceutical process. An example of such bioprocess is the use of a bioreactor to cultivate microorganisms or mammalian cells under given conditions.

The number of sensors and actuators connected to bioprocessing equipment has increased significantly due to several factors including regulatory pressure, cost pressure, process intensification, and flexibility requirements. This has created a “zoo” of connectors and signal types for digital, analog, and networked devices in the market.

While a number of plug and play concepts has been proposed, plug and play components using a standardized interface are not widely available. On the contrary, different bus systems are used today.

As new sensors are developed and sold, equipment must be upgraded with new hardware and software. Not only is this burdensome for the product development, but it is also problematic for customers since a field upgrade is not always possible and revalidation of equipment is costly and time-consuming. In the future, smart sensors could provide all information needed by the equipment without intervention from automation engineers and without triggering revalidation, thereby reducing cost and increasing the capability and flexibility of equipment.

The known prior art (EP 3 839 670 A1) that builds the basis of some embodiments is related to a method. This prior art teaches generally using plug and play components, reading functional information from those components and configuring a bioprocess based on this functional information. In general, process control systems in biotechnology use a digital model with digital representations of (replaceable) physical components for controlling the process, i.e. the physical components, and for communicating with a user.

On a more general level, for example IEEE 1451 defines possible ways of communication between components. Further, standards like Modbus are often implemented in process control systems and can be used. However, IEEE 1451 is not widely adapted in the field of biotechnology and some implementations may be too complex, in particular for cheap single-use components. Modbus is simple to implement but places the burden on the operator.

It is a challenge to improve plug and play capabilities in biotechnology.

Various embodiments are based on the problem of improving the known method such that the integration of plug and play components into workflows is made easier. A further objective is proposing components specifically well usable in the proposed method as plug and play components.

The above-noted object is solved by various features described herein.

A realization of some embodiments is that the production of bioproducts leads to frequent changes of electronic components, in particular single-use electronic components. Between batches of production or sometimes during continuous processes, electronic components need to be replaced. The replacement components can be essentially the same components. However, it is also possible that upgraded components are available and should be used. Due to these changes, digital representations of the physical components and the process control flow charts (a type of digital model) based on those digital representations come “out of sync” with the real world and need to be reconfigured. Usually, automatically reconfiguring the digital model is challenging as it might not be known well enough how a newly detected component should be used. In the field of biotechnology however, the change of components is a frequent and repetitive process. It is therefore easier to detect a new component and find a functional relationship with a previously removed component to identify that the new component is a replacement component. It is then possible to reconfigure the digital model automatically or to propose a reconfiguration to a user, such as in a way that the user just needs to confirm the correct detection.

In detail, it is proposed that the replaceable electronic components are removed during the bioprocess and/or between repetitions of the bioprocess and replaced by new electronic components, that the process control system automatically detects the removal of the replaceable electronic components, that the process control system automatically detects the presence of the new electronic components, that the process control system communicates with the new electronic components, that the process control system retrieves digital representations of the new electronic components based on the communication with the new electronic components, that the process control system derives from the digital representations of the replaceable electronic components and the new electronic components a functional relationship between the replaceable electronic components and the new electronic components, that the process control system replaces the digital representations of the replaceable electronic components with the digital representations of the new electronic components based on the functional relationship thereby updating the digital model, and, that the process control system controls the bioprocess arrangement to perform the bioprocess based on the updated digital model.

Various embodiments include new electronic components. In general, the new and the replaceable electronic components may be of the same type and everything said for the new electronic components may be true additionally for some or all replaceable electronic components. Nevertheless, in some embodiments, the new electronic components are upgraded electronic components and/or the replaceable electronic components are legacy components. In various embodiments, some electronic components are single-use components. Single-use electronic components should be as simple as possible to minimize cost, footprint, and environmental impact. The proposed protocols are designed to need only few resources and therefore particularly suitable for single-use electronic components.

Various embodiments relate to ways of communication between the process control system and the electronic components. It is proposed to use a communication protocol which allows the process control system to query memory sections, such as registers, of the electronic components. To allow the process control system to communicate with electronic components it does not know, it is proposed to use self-describing electronic components. It is therefore not necessary to update the process control system before using electronic components not used before.

The memory sections may vary between different new electronic components, but a description of the memory sections may be available at a standardized memory position.

The communication protocol can be Modbus or a similar protocol. Other protocols (e.g. Ethernet, CAN IOLink) have been evaluated and can be used, however show different disadvantages overcome by using Modbus.

Smart sensors will often lack Ethernet capability because of resource constraints and can therefore not support higher-level protocols such as HTTP(S) or OPC-UA. Ethernet is relatively costly to implement on sensor nodes because of the need for a processor capable of handling a TCP/IP stack, plus a MAC/PHY and the additional circuitry to deal with PoE or PoDL protocols for power distribution. Ethernet does not support multidrop without adding a 3-port switch to each device, further increasing the cost. Chips supporting new Single Pair Ethernet (SPE) protocols—in particular the Power over Data Line (PoDL) specifications—are not yet widely available.

Interfaces for CAN (including derivatives like CANopen and DeviceNet) are not available across all process control systems. Gateways are generally available to translate between native protocols and CAN, but these are typically costly and are not supported by the process control systems. IOLink does not have multidrop capability.

Wireless protocols have a low adoption rate of wireless communication in the bioprocessing industry and there is generally a low feasibility of long-range wireless power transmission for always-on devices. Advances have been made in RFID, SAW, and similar technologies, but there is no indication that these devices are ready for always-on sensor applications.

Various embodiments relate to the implementation of Modbus. A sensible selection is assigning the process control system as the client and the electronic components as servers. This selection also leads to possible backwards compatibility with legacy servers.

Various embodiments relate to details of the physical implementation.

Various embodiments describe an implementation of a plug and play capability on top of Modbus and how the Modbus servers and client may communicate. The proposed steps can be implemented even when legacy Modbus components are part of the network. Modbus does not support plug and play natively but can be tweaked, as proposed, to get plug and play, self-describing, low-cost electronic components.

Various embodiments focus on the client side and the bioprocess control. Various embodiments specify that the new and replaced electronic components may be of the same type or the new electronic components may be of a different type. For example, a manufacturer may have made a new model of a pH sensor and the new model can be plugged into a bus slot after removing the old sensor. The process control system, knowing that the current process needs a pH sensor, can automatically detect the new component, read its functional description from the corresponding memory section and then integrate the new pH sensor into the digital model without user intervention.

The new electronic components may be checked for compatibility automatically. The proposed method may also be used to scale up a bioprocess from a lower volume to a higher volume, in particular a final production volume. While a part of the system changes, the process control may be able to use the same or a slightly adapted digital model. A user may be asked for confirmation of the digital replacement to conform with regulatory requirements.

Various embodiments relate to an electronic component configured to be used as a new electronic component in and specifically designed for the proposed method.

All explanations given with regard to the proposed method are fully applicable.

Various embodiments relate to a process control system configured to be used in the proposed method.

All explanations given with regard to the proposed method and the proposed electronic component are fully applicable.

Various embodiments provide a method of operating a bioprocess arrangement to perform at least one repetition of a bioprocess, wherein the bioprocess arrangement comprises physical components for performing the bioprocess, wherein the physical components comprise at least one replaceable electronic component, wherein the bioprocess arrangement comprises a process control system, wherein the process control system controls the bioprocess arrangement, wherein the process control system uses a digital model of the bioprocess arrangement for controlling the bioprocess arrangement, wherein the digital model comprises digital representations of the replaceable electronic components, wherein the process control system communicates with the replaceable electronic components to perform the bioprocess by controlling the bioprocess arrangement based on the digital model, wherein the replaceable electronic components are removed during the bioprocess and/or between repetitions of the bioprocess and replaced by new electronic components, that the process control system automatically detects the removal of the replaceable electronic components, that the process control system automatically detects the presence of the new electronic components, that the process control system communicates with the new electronic components, that the process control system retrieves digital representations of the new electronic components based on the communication with the new electronic components, that the process control system derives from the digital representations of the replaceable electronic components and the new electronic components a functional relationship between the replaceable electronic components and the new electronic components, that the process control system replaces the digital representations of the replaceable electronic components with the digital representations of the new electronic components based on the functional relationship thereby updating the digital model, and, that the process control system controls the bioprocess arrangement to perform the bioprocess based on the updated digital model.

In various embodiments, the new electronic components comprise a sensor and/or an actuator and/or a single-use component, in particular a single-use sensor and/or a single-use actuator, and/or an electronic component in direct contact with a fluid used in the bioprocess and/or a component acting on or sensing a property of a fluid used in the bioprocess and/or a sterilizable multi-use component.

In various embodiments, the new electronic components comprise a memory, that the new electronic components save process data in a readable memory section, that the new electronic components communicate with the process control system via a communication protocol, that via the communication protocol the process control system can read memory sections, in particular registers, of the new electronic components, that via the communication protocol the new electronic components send a memory section description to the process control system, that the readable memory section is subdivided into information sections, that the subdivision of the memory sections is at least partially described by the memory section description and not by the communication protocol, that the process control system reads the readable memory section based on the memory section description.

In various embodiments, the subdivision varies between different new electronic components, and/or, that the memory section description is included in the readable memory section at a predefined location known to the process control system without knowledge of the memory section description.

In various embodiments, the communication protocol is a bus, such as a field bus or Modbus, and/or, that the process control system and the new electronic components communicate over a physical connection, in particular a physical connection providing power and signals. In some embodiments, the communication uses the RS-485 protocol.

In various embodiments, the process control system communicates as a Modbus client and the new electronic components communicate as Modbus servers. In various embodiments, the process control system is adapted to communicate with legacy Modbus electronic components in addition to the new electronic components.

In various embodiments, the physical connection between the process control system and the new electronic components comprises, in particular exactly, four individual conductors, in particular wires, and/or, that the topology of the physical connection is a daisy chain multi-drop topology or a point-to-point topology.

In various embodiments, the new electronic components are automatically discovered by the process control system, that for discovering the new electronic components the process control system performs a discovery routine and the new electronic components perform an initialization routine. In various embodiments, during the discovery routine the process control system sets addresses for the new electronic components and during the initialization routine the new electronic components set the addresses as their addresses.

In various embodiments, during the discovery routine the process control system broadcasts an initialization message, in particular an empty write request, and then sends a read request at an address not yet assigned, that the new electronic components respond to a read request at an address not assigned to them after detecting a broadcasted initialization message.

In various embodiments, during the discovery routine the process control system addresses multiple, in particular all, new electronic components that have not performed the discovery routine at once with an initialization request, in particular a Modbus slot read request. In some embodiments, the initialization request contains one of several possible initialization IDs, in particular Modbus slot numbers as part of the Modbus slot read request. In various embodiments, the new electronic components have an assigned, in particular random, initialization ID, in particular Modbus slot number, that of the addressed new electronic components only those with a matching initialization ID perform the discovery routine.

In various embodiments, the process control system iterates through the possible initialization IDs, and/or, that if several new electronic components respond to the initialization request at once, the process control system repeats the initialization request and the responding new electronic components follow a collision avoidance strategy, in particular by adding random delays to the repeated responses.

In various embodiments, the new electronic components set a baud rate for the communication by cycling through baud rates, and/or, that the new electronic components use the Modbus 8N1 serial format.

In various embodiments, at least one replaceable electronic component and at least one new electronic component replacing the replaceable electronic component in the bioprocess are of the same model, and/or, that at least one replaceable electronic component and at least one new electronic component replacing the replaceable electronic component in the bioprocess are of a different model, and/or, that at least one new electronic component does not replace any of the replaceable electronic components.

In various embodiments, the process control system receives from the new electronic components compatibility data and checks the compatibility of the new electronic components with other present electronic components and/or the process control system and/or the bioprocess, and/or, that the process control system receives from the new electronic components documentation data, in particular validation data, and documents the replacement of the electronic components.

In various embodiments, several electronic components are replaced at the same time together with other single-use components, in particular between repetitions of the bioprocess. In various embodiments, the repetitions of the bioprocess comprise repetitions with different, in particular increasing, liquid volumes.

In various embodiments, the process control system queries a user prior to replacing the digital representations of the replaceable electronic components with the digital representations of the new electronic components.

Various embodiments provide an electronic component configured to be used in the method according to the disclosure as a new electronic component.

Various embodiments provide a process control system configured to be used in the method according to the disclosure.

### BRIEF DESCRIPTION OF THE DRAWINGS

In the following, embodiments are explained with respect to the drawing. The drawing shows in

FIG. 1, a bioprocess arrangement with a process control system and a bioreactor,

FIG. 2, the process control system of FIG. 1 connected to a scaled-up bioreactor asking the user for confirmation of the replacement of digital representations of electronic components,

FIG. 3, schematically the updating of the digital model (from a) to b)) and

FIG. 4, a connector for the proposed system and a possible system topography

### DETAILED DESCRIPTION

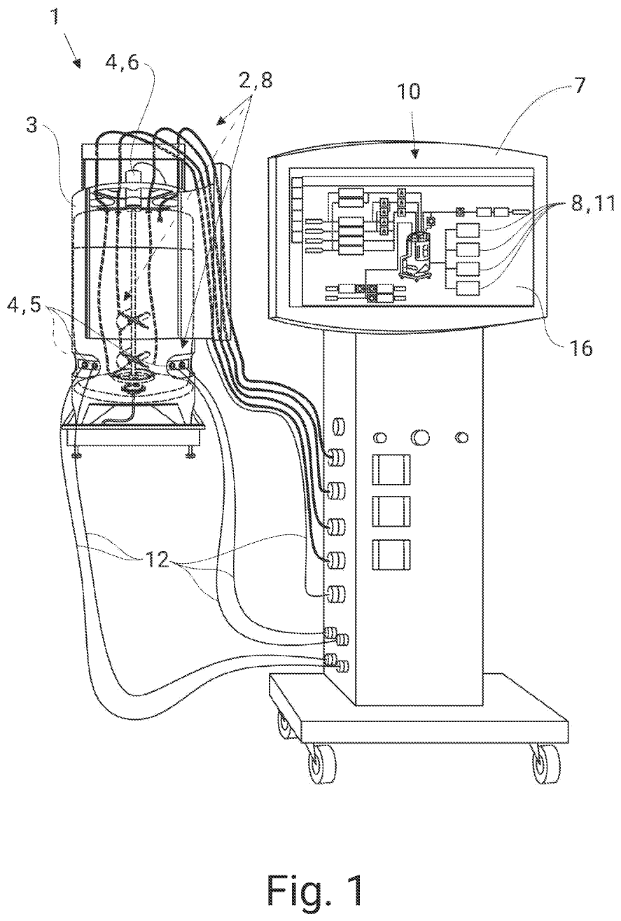

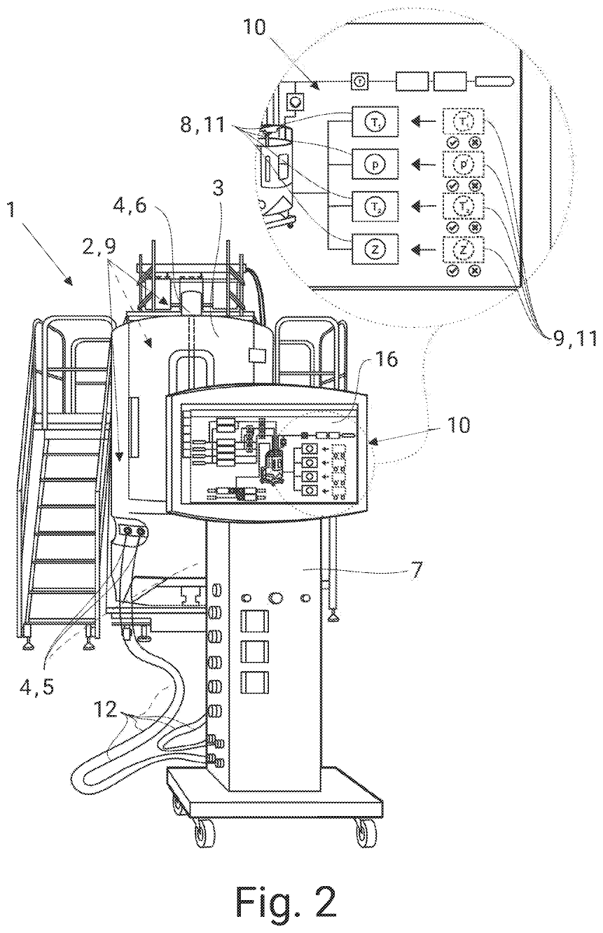

FIGS. 1 and 2 show bioprocess arrangements 1 with physical components 2 including a bioreactor 3. The bioreactor 3 in FIG. 2 is considerably larger than the bioreactor 3 in FIG. 1. Such a scale-up is often done during the development of production processes and a good example use-case for the proposed method. The physical components 2 also include several electronic components 4 like sensors 5, mixers 6, etc. Further shown is a process control system 7, which in FIG. 1 and FIG. 2 is the same process control system 7. Being able to use the same process control system 7 for different scaling stages is generally advantageous. It would be desirable to also use the same electronic components 4, however, that is usually not feasible. Some electronic components 4 will be single-use and others, even if not single-use, cannot be used with a larger system.

After having reached production size of the bioprocess arrangement 1, the bioprocess arrangement 1 needs regular replacement of electronic components 4, either between repetitions or, in particular for continuous processes, during production. It will now be described how these changes and replacements of electrical components can be handled efficiently by the process control system 7.

Proposed is a method of operating a bioprocess arrangement 1 to perform at least one repetition of a, in particular batch or continuous, bioprocess. The bioprocess arrangement 1 comprises physical components 2 for performing the bioprocess which comprise at least one replaceable electronic component 8. Basically every electronic component 4 is replaceable, the term is used here to differentiate between electronic components 4 at the beginning of an iteration of the proposed method and new electronic components 9 which are introduced during the execution of the proposed method. The new electronic components 9 may be of the same type as the replaceable electronic components 8 and may serve as replaceable electronic components 8 during another iteration of the method. The new electronic components 9 may however also be upgrades or model changes of the replaceable electronic components 8.

The bioprocess arrangement 1 comprises a process control system 7, e.g. a Sartorius Biobrain. The process control system 7 controls the bioprocess arrangement 1. The process control system 7 may measure process parameters and influence process parameters through actuators.

The process control system 7 uses a digital model 10 of the bioprocess arrangement 1 for controlling the bioprocess arrangement 1. Digital models 10 as used for process flow charts are all types of models in which knowledge about specific components is used to control the bioprocess arrangement 1. The term “model” is therefore to be understood broadly. Any representation of a functional relationship between components or between a component and the bioprocess is seen as a model.

The digital model 10 comprises digital representations 11 of the replaceable electronic components 8. The term “digital representations” is also to be understood broadly as any digital information about the electronic components 4 that can be used for the control of the bioprocess. It is not necessary for the digital model 10 to comprise all electronic and/or physical components 2 of the bioprocess arrangement 1.

The process control system 7 communicates with the replaceable electronic components 8 to perform the bioprocess by controlling the bioprocess arrangement 1 based on the digital model 10.

Proposed is that the replaceable electronic components 8 are removed during the bioprocess and/or between repetitions of the bioprocess and replaced by new electronic components 9. The repetitions may be repetitions of the same or a scaled up bioprocess. The new electronic components 9 are one or more components. The plural will be used here, but it may be the case that only a single component is actually present.

The process control system 7 automatically detects the removal of the replaceable electronic components 8, in particular by detecting that a communication is no longer possible. The process control system 7 automatically detects the presence of the new electronic components 9. The process control system 7 communicates with the new electronic components 9, and retrieves digital representations 11 of the new electronic components 9 based on the communication with the new electronic components 9. “Based on the communication” means that any information retrieved from the communication is used to retrieve the digital representation 11. In particular, the digital representation 11 may be partially sent by the new electronic components 9 but also stored in a memory of the process control system 7 or retrieved from the internet. In various embodiments, the digital representation 11 is completely retrieved from the new electronic components 9.

The process control system 7 derives from the digital representations 11 of the replaceable electronic components 8 and the new electronic components 9 a functional relationship between the replaceable electronic components 8 and the new electronic components 9. The functional relationship may for example be that both components are temperature sensors 5 or that both sensors 5 are of the same type.

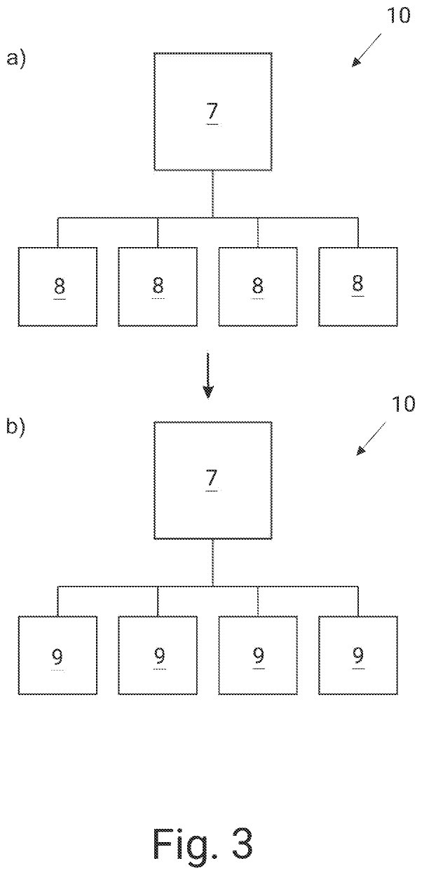

The process control system 7 replaces the digital representations 11 of the replaceable electronic components 8 with the digital representations 11 of the new electronic components 9 based on the functional relationship thereby updating the digital model 10. The process control system 7 controls the bioprocess arrangement 1 to perform the bioprocess based on the updated digital model 10. For example, a user may connect a new type of temperature sensor 5 for replacing an old temperature sensor 5 and the process control system 7 automatically detects this change and is able to cope with it. FIG. 3 shows very schematically a change of the digital model 10.

The new electronic components 9 may comprise a sensor 5 and/or an actuator and/or a single-use component, in particular a single-use sensor 5 and/or a single-use actuator, and/or an electronic component 4 in direct contact with a fluid used in the bioprocess, e.g. a stirrer, and/or a component acting on or sensing a property of a fluid used in the bioprocess, e.g. a pH sensor 5, temperature sensor 5, cell mass sensor 5 or the like, and/or a sterilizable multi-use component. Everything said for new electronic components 9 may be true for the replaceable electronic components 8, too. However, it is also conceivable to upgrade from legacy replaceable components without much of the capabilities of the new electronic components 9 to new electronic components 9.

In the following, details of various modes of communication between the process control system 7 and the new electronic components 9 will be explained. These may apply to some or all replaceable electronic components 8. In various embodiments, the new electronic components 9 are self-describing. It is therefore possible for the process control system 7 to retrieve the digital representation 11 of a new electronic component 9 from the new electronic component 9, in particular by applying information sent from the new electronic component 9 to a template which is independent of the specific new electronic component 9, in particular a template used for all new electronic components 9.

It can be that the new electronic components 9 comprise a memory. The memory may comprise writable and read-only sections. Other organizations of the memory are also possible. The new electronic components 9 may save process data in a readable memory section.

The new electronic components 9 may communicate with the process control system 7 via a communication protocol, such as Modbus. Via the communication protocol the process control system 7 can read memory sections, in particular registers, of the new electronic components 9.

The new electronic components 9 may be adapted to send, via the communication protocol, a memory section description to the process control system 7. It may be the case that all new electronic components 9 comprise a memory section located at the same memory address which contains or leads to a description of other sections of the memory. Hence, the new electronic components 9 may be self-describing. The process control system 7 may read this memory section and apply a common template to understand what information the new electronic component 9 can provide and how to access this information. The memory may contain information like measurement readings, units, preferred modes of displaying the measurements, possible performable actions, or editable settings and so on. The memory of different new electronic components 9, e.g. actuators and sensors 5, may be structured differently, however, through the memory section description the process control system 7 can access the memory sections without previous knowledge about the new electronic component 9.

The readable memory section can then be subdivided into information sections. The subdivision of the memory sections is at least partially described by the memory section description and not by the communication protocol. The process control system 7 therefore reads the readable memory section based on the memory section description. Read commands used by the process control system 7 may be part of the communication protocol as is the case with Modbus. It should be understood that all descriptions of the communication protocol, in particular the described tweaks to Modbus, are relevant on their own merits independently of the described use by the process control system 7 or the electronic components 4. It is proposed to implement a self-describing data structure on top of the communication protocol, in particular on top of Modbus.

Generally, it can be that the subdivision varies between different new electronic components 9, and/or, that the memory section description is included in the readable memory section at a predefined location known to the process control system 7 without knowledge of the memory section description. The process control system 7 may use a discovery routine to detect new electronic components 9 and/or learn their memory organization.

In the following, an implementation on top of Modbus is described. Modbus defines 65536 holding registers numbered 0 to 65535. In this document all register addresses are 0-based. Absolute addresses refer to the range 0 to 65535, and relative addresses are offsets with respect to the start of the table in which the address is contained. Each holding register is 16 bits. Modbus function codes 3 and 16 permit reading or writing multiple holding registers in a single request/response transaction. The proposed protocol defines multiple tables. An Identification Table can always start at register 0. The location for all other tables is at the discretion of the server manufacturer. Table locations and lengths may be constant, i.e. they shall not change unless firmware changes.

A Dynamic Table is the only table in which the data can change. All other tables and strings may be read-only and constant, i.e. they shall not change unless firmware changes. This permits servers to store “wire ready” tables which are ready for sending without any modification. Clients can read and cache the content of these constant tables without risk of them changing unexpectedly.

Clients may use a combination of a manufacturer ID, a device ID, and a firmware version to cache constant information in other tables. If the table layout changes, it may be mandatory to increment a firmware version. Information stored in the Identification Table may be a Magic ID, a protocol version, a manufacturer ID, a device ID, a serial number, which may be unique for the given manufacturer ID and device ID, a hardware version, a firmware version, a main table start, a main table length and/or a main table CRC as a means of checking corruption.

Summarized and additionally, it can be that the communication protocol is a bus, such as a field bus or Modbus, and/or, that the process control system 7 and the new electronic components 9 communicate over a physical connection 12, in particular a, in particular single cable, physical connection 12 providing power and signals. In some embodiments, the communication uses the RS-485 protocol. Here, RS-485 as specified in ANSI/TIA/EIA-485-A-1998 is meant.

All electronic components 4 on the bus may support a subset of the Modbus protocol including RTU framing (MODBUS/RTU), function code 3 (read holding registers), function code 16 (write multiple registers), and exceptions. Here, Modbus refers to the documents Modbus Protocol Specification V1.1b3 and Modbus Serial Line Protocol and Implementation Guide V1.02.

According to one embodiment it is proposed that the process control system 7 communicates as a Modbus client and the new electronic components 9 communicate as Modbus servers. In some embodiments, the process control system 7 is adapted to communicate with legacy Modbus electronic components 4 in addition to the new electronic components 9. The legacy Modbus electronic components 4 may be present on the same physical connection 12 without providing plug and play capabilities.

In various embodiments, if a client sends a message to a server and no response is received, the client will retry up to two times; if the server does not respond then the client may consider it to be non-responsive.

In various embodiments, the process control uses an empty, in particular zero registers and zero bytes, function code 16 request as a control channel for the client to communicate information to all servers, in particular using broadcast address 0, or to a specific server (using its assigned address). The register address of the write is interpreted by servers with specific meanings and ignored as a no-op by legacy MODBUS/RTU servers.

There may be only one client device on the bus, and it can generally be a process control system 7 or an embedded controller. The client may provide 24 VDC power to all server devices. The client may initiate all communication. The client may not have an address. There may be up to 32 server devices on the bus. Servers may consume 24 VDC power provided by the client. Servers may not initiate communication; they may only respond to client requests. Each server may have a unique address.

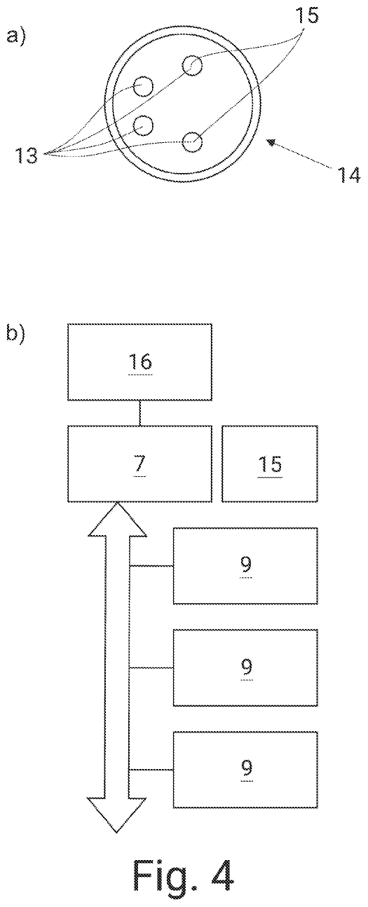

According to one embodiment it is proposed that the physical connection 12 between the process control system 7 and the new electronic components 9 comprises, in particular exactly, four individual conductors 13, in particular wires, (shown exemplarily in FIG. 4) and/or, that the topology of the physical connection 12 is a daisy chain multi-drop topology or a point-to-point topology. FIG. 4 shows an architecture of the proposed physical connection 12 with several electronic components 4, a view onto a connector 14 (here equaling a cross-section) of the physical connection 12, a 24 VDC supply 15, a user interface 16 and the process control system 7.

In various embodiments, four pin IP 67 or higher connectors 14 are used where the client may comprise female contacts and the servers male. The pins may be organized with pins A and B from RS-485, 24 VDC/range 18-30 VDC, max. 1 A, GND and shield connected to ground. Cables may be shielded twisted pair (STP) with at least 2 pairs.

The last device at each end in a daisy-chain may implement a terminating resistor. Electronic components 4 and/or the process control system 7 may have a fail-safe feature in their RS-485 transceiver to deal with the case that no drivers are active on the network. Legacy RS-485 devices may require an interposing adapter to provide this functionality.

In various embodiments, the new electronic components 9 are automatically discovered by the process control system 7. For discovering the new electronic components 9 the process control system 7 may perform a discovery routine and the new electronic components 9 may perform an initialization routine. In various embodiments, during the discovery routine the process control system 7 sets addresses for the new electronic components 9 and during the initialization routine the new electronic components 9 set the addresses as their addresses.

In various embodiments, when first starting, the client goes through several tasks:
Broadcast empty write to holding register MSG_KEEPALIVE every 100 ms for 3 s
Check legacy servers at reserved fixed addresses
Discover servers and assign addresses
Broadcast empty write to holding register MSG_ONLINE

To discover legacy servers at reserved fixed addresses, the client sequentially reads 1 holding register at address 0 for the reserved fixed server addresses (if any). New electronic components 9 as servers must not respond since they do not have an address yet. Legacy servers will respond either with a normal response or an exception response—it doesn't matter as long as the “ping” returns. In this way the client checks connectivity to any legacy servers present.

According to one embodiment it is proposed that during the discovery routine the process control system 7 broadcasts an initialization message, in particular an empty write request, and then sends a read request at an address not yet assigned. The new electronic components 9 respond to a read request at an address not assigned to them after detecting a broadcasted initialization message.

It may be the case that during the discovery routine the process control system 7 addresses multiple, in particular all, new electronic components 9 that have not performed the discovery routine at once with an initialization request, in particular a Modbus slot read request. In various embodiments, the initialization request contains one of several possible initialization IDs, in particular Modbus slot numbers as part of the Modbus slot read request, and the new electronic components 9 have an assigned, in particular random, initialization ID, in particular Modbus slot number. Of the addressed new electronic components 9 only those with a matching initialization ID perform the discovery routine.

The discovery routine may be run on network power-up and at intervals at the discretion of the client. In this way the client becomes aware of new servers. An embodiment of a discovery routine is now described. When a server powers up, it has no address (ignoring 247 and broadcast) and assigns itself to one of several, e.g. 32, “slots” using for example the least significant 5 bits of its serial number. This significantly reduces and may possibly eliminate bus contention, especially where only a few devices are present. For each slot, the client broadcasts an empty write to holding register MSG_SLOTx (where x is the slot number) and then reads ManufacturerID+DeviceID+SerialNumber found in holding registers 2-7 (six registers=96 bits) on the next unoccupied server address such as starting at 1. There are 3 outcomes:

1. No servers are in the slot, no response is received by the client, so a timeout occurs. The client skips to the next slot.

2. One server is in the slot, and a correctly formatted response is received by the client. The client writes ManufacturerID+DeviceID+SerialNumber back to holding registers 2-7 on the unoccupied server address. The server checks for a match with its ManufacturerID+DeviceID+SerialNumber and if there is a match then it takes the server address and sends a normal write response. The correct response from the server indicates that the address is successfully assigned, so the client goes to the next unoccupied server address and retries. If no response is received, the client goes to the next slot.

3. Multiple servers are in the slot, so a garbled response (bad CRC) is received. Exponential backoff is then used to get clear reads between the contending devices. The client extends its timeout to 1 s. The client retransmits the slot number and each server randomly adds a delay of 0 ms or 1 ms. If garbled again, the client retransmits the slot number and each server randomly adds a delay of 0 ms, 1 ms, or 2 ms. If garbled again, the client retransmits the slot number and each server randomly adds a delay of 0 ms, 1 ms, 2 ms, 3 ms, or 4 ms. This continues in the sequence 1, 2, 4, 8, . . . 512. This possibly continues until the client receives a clear response. Once a clear response is received, the client returns to its usual 100 ms timeout. Then the client goes to the next available server address and tries again until no responses are received. Then the client goes to the next slot.

Since this is a lengthy operation lasting seconds, it is not convenient to interrupt normal network traffic after initial configuration. The client can interleave discovery routines with other traffic at low priority. The client may send an empty write to holding register MSG_RELEASEADDR at a single server address forcing it to abandon its automatically assigned address. The client may broadcast an empty write to holding register MSG_RELEASEADDR forcing all servers to abandon their automatically assigned addresses. For the case that a legacy client needs to configure a server into a new server address that is retained through power losses, a client may send an empty write to holding register MSG_RETAINADDR at a single server address. The automatically assigned server address is used until the server receives a MSG_RELEASEADDR message.

More generally described, according to one embodiment it is proposed that the process control system 7 iterates through the possible initialization IDs, and/or, that if several new electronic components 9 respond to the initialization request at once, the process control system 7 repeats the initialization request and the responding new electronic components 9 follow a collision avoidance strategy, in particular by adding random delays to the repeated responses.

According to one embodiment it is proposed that the new electronic components 9 set a baud rate for the communication by cycling through baud rates, and/or, that the new electronic components 9 use the Modbus 8N1 serial format.

In legacy MODBUS/RTU over RS-485 networks, each device has three settings that must be configured before communication can begin:
Data rate—also known as baud rate, measured in bits per second (bps) and typically one of 9600, 19200, 38400, or 57600 bps. The data rate could be any value supported by the cable type, cable length, and UART capabilities. The Modbus serial spec requires support for 9600 and 19200 bps (default).
Serial format—number of data bits, parity type, and number of stop bits. The Modbus serial spec requires 8E1 and recommends 8N2 to keep an 11-bit frame.
Address—each server must have a unique address.

Configuring these settings can be highly challenging on legacy devices because they may require special software, adapter cables, DIP switch settings, etc.

To achieve plug and play capability, the proposed bus in one embodiment simplifies by flexibility:
Data rate—client defaults to 115200 bps but may be overridden to 9600 or 19200 bps for compatibility with legacy servers. Servers must auto-detect the data rate at power-up by first looking for “magic frames” from MSG_KEEPALIVE corruptions due to baud rate being wrong, then by cycling through baud rates until a well-formed packet arrives.
Serial format—fixed at 8 data bits, no parity, 1 stop bit (8N1) as this is the format typically used for industrial serial communication. Any legacy server that cannot be configured for 8N1 will need to use an adapter or a separate bus. This setting creates a 10-bit frame; although this is, strictly speaking, a deviation from the Modbus serial specification, it is common practice in industrial settings since parity adds little value and the frame integrity is already protected by a CRC.
Address—the client automatically assigns available addresses to servers, optionally reserving fixed addresses for legacy servers.

The Modbus protocol specifies a minimum inter-frame delay but does not specify a maximum inter-frame delay. Since the time between frames is a key driver for overall frame rate, the proposed protocol in one embodiment establishes additional timing constraints. Timeout value for the client (i.e. if no characters are received within this time then it may retry or consider a server to be non-responsive) may be set at 10 ms unless slower legacy servers are present. There is a processing window between the moment when the request frame end can be detected (t1.5 elapsed) and the moment when the response frame transmission can start (t3.5 elapsed); at 9600 bps it is 2.08 ms, at 19200 bps it is 1.04 ms, and at higher data rates it is 1.00 ms. Server devices must start transmitting a response no more than 5 ms after the last character of the request is received. Servers must have adequate processing power and efficient execution flow (e.g. using interrupts instead of blocking calls) to keep up with the pace. Initial feasibility tests with an ARM Cortex-M0+ processor running FreeRTOS with a system tick frequency of 1 ms indicate that it is easily able to keep the processing window less than 2 ms.

Turning back towards the more general process view, it can be that at least one replaceable electronic component 8 and at least one new electronic component 9 replacing the replaceable electronic component 8 in the bioprocess are of the same model, i.e. generally the same, and/or, that at least one replaceable electronic component 8 and at least one new electronic component 9 replacing the replaceable electronic component 8 in the bioprocess are of a different model, and/or, that at least one, additional, new electronic component 9 does not replace any of the replaceable electronic components 8. The proposed embodiments are particularly suited for replacing replaceable electronic components 8, e.g. a temperature sensor 5, with newer, more capable, more precise or the like, new electronic components 9, even from other manufacturers.

According to various embodiments, the process control system 7 receives from the new electronic components 9 compatibility data, for example functional data, limits, calibration data or the like, and checks the compatibility of the new electronic components 9 with other present electronic components 4 and/or the process control system 7 and/or the bioprocess. Additionally or alternatively, it may be the case that the process control system 7 receives from the new electronic components 9 documentation data, in particular validation data, and documents the replacement of the electronic components 4. Keeping an audit trail may be particularly important in the present field. Automatically supporting this audit trail therefore is advantageous. It may additionally or alternatively be the case that the process control system 7 receives from the new electronic components 9 graphical user interface data relating to the presentation of data received from the new electronic components 9 and/or documents and/or links to documents and/or data in multiple languages.

According to one embodiment it is proposed that several electronic components 4 are replaced at the same time together with other single-use components, in particular between repetitions of the bioprocess. For example, a single-use bioreactor 3 with several sensors 5 and actuators may be replaced at once. In various embodiments and as demonstrated between FIGS. 1 and 2, the repetitions of the bioprocess may comprise repetitions with different, in particular increasing, liquid volumes.

According to one embodiment it is proposed that the process control system 7 queries a user prior to replacing the digital representations 11 of the replaceable electronic components 8 with the digital representations 11 of the new electronic components 9.

For example, a single-use bioreactor 3 with a set of sensors 5 is removed, a new bioreactor 3 is implemented, some sensors 5 are exchanged for new single use and/or sterilized multi use sensors 5, either of the same model or of a similar model, the digital control model is automatically updated and the user can accept the automatic detection and start a new iteration with one click.

Another teaching which is of equal importance relates to an electronic component 4 configured to be used in the proposed method as a new electronic component 9. The electronic component 4 may have a processor and a memory and possibly no other communication capability than the proposed one.

All explanations given with regard to the proposed method are fully applicable.

Another teaching which is of equal importance relates to a process control system 7 configured to be used in the proposed method.

All explanations given with regard to the proposed method and the proposed electronic component 4 are fully applicable.

1. A method of operating a bioprocess arrangement to perform at least one repetition of a bioprocess, wherein the bioprocess arrangement comprises physical components for performing the bioprocess, wherein the physical components comprise at least one replaceable electronic component, wherein the bioprocess arrangement comprises a process control system, wherein the process control system controls the bioprocess arrangement,
wherein the process control system uses a digital model of the bioprocess arrangement for controlling the bioprocess arrangement, wherein the digital model comprises digital representations of the replaceable electronic components, wherein the process control system communicates with the replaceable electronic components to perform the bioprocess by controlling the bioprocess arrangement based on the digital model,
wherein the replaceable electronic components are removed during the bioprocess and/or between repetitions of the bioprocess and replaced by new electronic components, that the process control system automatically detects the removal of the replaceable electronic components, that the process control system automatically detects the presence of the new electronic components, that the process control system communicates with the new electronic components, that the process control system retrieves digital representations of the new electronic components based on the communication with the new electronic components, that the process control system derives from the digital representations of the replaceable electronic components and the new electronic components a functional relationship between the replaceable electronic components and the new electronic components, that the process control system replaces the digital representations of the replaceable electronic components with the digital representations of the new electronic components based on the functional relationship thereby updating the digital model, and, that the process control system controls the bioprocess arrangement to perform the bioprocess based on the updated digital model.

2. The method according to claim 1, wherein the new electronic components comprise a sensor and/or an actuator and/or a single-use component, in particular a single-use sensor and/or a single-use actuator, and/or an electronic component in direct contact with a fluid used in the bioprocess and/or a component acting on or sensing a property of a fluid used in the bioprocess and/or a sterilizable multi-use component.

3. The method according to claim 1, wherein the new electronic components comprise a memory, that the new electronic components save process data in a readable memory section, that the new electronic components communicate with the process control system via a communication protocol, that via the communication protocol the process control system can read memory sections, in particular registers, of the new electronic components, that via the communication protocol the new electronic components send a memory section description to the process control system, that the readable memory section is subdivided into information sections, that the subdivision of the memory sections is at least partially described by the memory section description and not by the communication protocol, that the process control system reads the readable memory section based on the memory section description.

4. The method according to claim 3, wherein the subdivision varies between different new electronic components, and/or, that the memory section description is included in the readable memory section at a predefined location known to the process control system without knowledge of the memory section description.

5. The method according to claim 3, wherein the communication protocol is a bus and/or, that the process control system and the new electronic components communicate over a physical connection.

6. The method according to claim 5, wherein the process control system communicates as a Modbus client and the new electronic components communicate as Modbus servers.

7. The method according to claim 5, wherein the physical connection between the process control system and the new electronic components comprises four individual conductors, that the topology of the physical connection is a daisy chain multi-drop topology or a point-to-point topology.

8. The method according to claim 5, wherein the new electronic components are automatically discovered by the process control system, that for discovering the new electronic components the process control system performs a discovery routine and the new electronic components perform an initialization routine.

9. The method according to claim 8, wherein during the discovery routine the process control system broadcasts an initialization message, in particular an empty write request, and then sends a read request at an address not yet assigned, that the new electronic components respond to a read request at an address not assigned to them after detecting a broadcasted initialization message.

10. The method according to claim 8, wherein during the discovery routine the process control system addresses multiple new electronic components that have not performed the discovery routine at once with an initialization request, wherein the initialization request contains one of several possible initialization IDs, and, that the new electronic components have an assigned initialization ID that of the addressed new electronic components only those with a matching initialization ID perform the discovery routine.

11. The method according to claim 10, wherein the process control system iterates through the possible initialization IDs, and/or, that if several new electronic components respond to the initialization request at once, the process control system repeats the initialization request and the responding new electronic components follow a collision avoidance strategy.

12. The method according to claim 5, wherein the new electronic components set a baud rate for the communication by cycling through baud rates, and/or, that the new electronic components use the Modbus 8N1 serial format.

13. The method according to claim 1, wherein at least one replaceable electronic component and at least one new electronic component replacing the replaceable electronic component in the bioprocess are of the same model, and/or, that at least one replaceable electronic component and at least one new electronic component replacing the replaceable electronic component in the bioprocess are of a different model, and/or, that at least one new electronic component does not replace any of the replaceable electronic components.

14. The method according to claim 1, wherein the process control system receives from the new electronic components compatibility data and checks the compatibility of the new electronic components with other present electronic components and/or the process control system and/or the bioprocess, and/or, that the process control system receives from the new electronic components documentation data, in particular validation data, and documents the replacement of the electronic components.

15. The method according to claim 1, wherein several electronic components are replaced at the same time together with other single-use components, wherein the repetitions of the bioprocess comprise repetitions with different liquid volumes.

16. The method according to claim 1, wherein the process control system queries a user prior to replacing the digital representations of the replaceable electronic components with the digital representations of the new electronic components.

17. An electronic component configured to be used in the method according to claim 3 as a new electronic component.

18. A process control system configured to be used in the method according to claim 1.

19. The method according to claim 3, wherein the communication protocol is a Modbus, and/or, that the process control system and the new electronic components communicate over a physical connection providing power and signals, wherein the communication uses the RS-485 protocol.

20. Method according to claim 19, wherein the process control system communicates as a Modbus client and the new electronic components communicate as Modbus servers, wherein the process control system is adapted to communicate with legacy Modbus electronic components in addition to the new electronic components.
